(19) United States
(12) Patent Application Publication
(10) Pub. No.: US 2026/0294820 A1
(43) Pub. Date: Oct. 1, 2026

(54) HYDROGEL AND OREXIN RECEPTOR LIGAND COMPOSITIONS AND METHODS OF MAKING AND USING THE SAME

(71) Applicants: Wake Forest University Health Sciences, Winston-Salem, NC (US); Wake Forest University, Winston-Salem, NC (US); Biologic Delivery Technologies LLC, Reno, NV (US)

(72) Inventors: Emmanuel C. Opara, Durham, NC (US); Mark E. Welker, Clemmons, NC (US); Donald James Kyle, Las Vegas, NV (US)

(21) Appl. No.: 19/480,634

(22) PCT Filed: May 7, 2024
(86) PCT No.: PCT/US2024/028087

§ 371 Date: Oct. 31, 2025

Related U.S. Application Data
(60) Provisional application No. 63/500,719, filed on May 8, 2023.

(51) Int. Cl. A61K 9/50; A61K 45/06

(52) U.S. Cl. CPC A61K 9/5036; A61K 9/5089; A61K 45/06

(57) ABSTRACT

Described herein are compositions including a hydrogel and an orexin receptor ligand. Also described herein are compositions including a hydrogel and a protease inhibitor. Further provided herein are compositions including a hydrogel, an orexin receptor ligand, and a protease inhibitor, wherein the orexin receptor ligand and the protease inhibitor are in the same or different hydrogels. Also provided herein are methods for making and/or using compositions of the present invention.

### RELATED APPLICATION INFORMATION

This application claims the benefit of U.S. Provisional Application Ser. No. 63/500,719, filed May 8, 2023, the disclosure of which is incorporated herein by reference in its entirety.

### FIELD

The present invention relates to compositions comprising a hydrogel and an orexin receptor ligand. Also provided are methods for making and/or using compositions of the present invention.

### BACKGROUND

The orexins, such as orexin-A and orexin-B, are neuropeptides that are known to play a role in regulating the sleep/wake cycle, reward systems, appetite control, and energy homeostasis. These peptides act by binding to two specific groups of G protein-coupled receptors (GPCRs), orexin receptor type 1 (OX1R) and orexin receptor type 2 (OX2R). In addition to this role in behavioral regulation, orexins are also able to promote apoptosis in various cancer cell lines and tumors that express OX1R and/or OX2R. Modulating level of orexins or the activity of the orexin receptors has garnered interest as a potential therapeutic option for sleep disorders, addiction, obesity, and cancer. Treatments with different orexin peptides and orexin receptor antagonists, also called orexin antagonists, have been shown to help treat insomnia and cancer.

### SUMMARY

A first aspect of the present invention is directed to a composition comprising a first hydrogel and an orexin receptor ligand. In some embodiments, the composition includes a protease inhibitor, optionally wherein the protease inhibitor is in the first hydrogel and/or a second hydrogel. In some embodiments, the orexin receptor ligand is orexin (e.g., orexin-A and/or orexin-B). In some embodiments, the orexin receptor ligand is an orexin antagonist, optionally wherein the orexin receptor ligand is almorexant. In some embodiments, the composition further comprises a therapeutic (e.g., a biologic, chemotherapeutic, and/or immunotherapeutic).

Another aspect of the present invention is directed to a method for treating a subject in need thereof, the method comprising administering a first hydrogel comprising an orexin receptor ligand to the subject, thereby treating the subject. In some embodiments, the method further comprises administering a protease inhibitor, optionally wherein the protease inhibitor is in the first hydrogel and/or a second hydrogel. In some embodiments, administering the composition comprises concurrently administering the first hydrogel and the protease inhibitor to the subject. In some embodiments, the first hydrogel and the protease inhibitor are present in the same composition. In some embodiments, the first hydrogel and the protease inhibitor are present in separate compositions. In some embodiments, the administering further comprises administering a therapeutic (e.g., a biologic, chemotherapeutic, and/or immunotherapeutic) to the subject.

It is noted that aspects of the present invention described with respect to one embodiment, may be incorporated in a different embodiment although not specifically described relative thereto. That is, all embodiments and/or features of any embodiment can be combined in any way and/or combination. Applicant reserves the right to change any originally filed claim and/or file any new claim accordingly, including the right to be able to amend any originally filed claim to depend from and/or incorporate any feature of any other claim or claims although not originally claimed in that manner. These and other objects and/or aspects of the present invention are explained in detail in the specification set forth below. Further features, advantages and details of the present invention will be appreciated by those of ordinary skill in the art from a reading of the figures and the detailed description of the preferred embodiments that follow, such description being merely illustrative of the present invention.

### BRIEF DESCRIPTION OF THE DRAWINGS

FIG. 1 is a graph showing the release profile of orexin encapsulated in hydrogel beads, as a percentage of the total encapsulated orexin released, over 28 days.

FIG. 2 is a graph showing the release profile of orexin encapsulated in hydrogel beads, as a cumulative amount (ng) of released orexin, over 28 days.

FIG. 3A is a graph showing the tumor volume in athymic nude mice inoculated with ASPC1 pancreatic tumor cells over 36 days for four different treatments (black arrows show day of treatment) of either phosphate buffered saline (PBS; Control), empty hydrogel beads (Blank-Beads), free orexin, or orexin encapsulated in hydrogel beads (Orexin-Beads).

FIG. 3B is a graph showing the tumor volume on the day of mice sacrifice (day 36).

FIG. 4 is a graph showing the amount of cancer antigen 19-9 (CA 19-9) in blood samples from athymic nude mice on day 36 after inoculation with ASPC1 pancreatic tumor cells and after 21 days of treatment with either orexin encapsulated in hydrogel beads or PBS (control); * indicates a P-value<0.05.

### DETAILED DESCRIPTION

The present invention is now described more fully hereinafter with reference to the accompanying drawings, in which embodiments of the invention are shown. This invention may, however, be embodied in many different forms and should not be construed as limited to the embodiments set forth herein; rather these embodiments are provided so that this disclosure will be thorough and complete and will fully convey the scope of the invention to those skilled in the art.

The terminology used in the description of the invention herein is for the purpose of describing particular embodiments only and is not intended to be limiting of the invention. As used in the description of the invention and the appended claims, the singular forms “a,” “an” and “the” are intended to include the plural forms as well, unless the context clearly indicates otherwise.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein.

All publications, patent applications, patents and other references mentioned herein are incorporated by reference in their entirety. In case of a conflict in terminology, the present specification is controlling.

Also as used herein, “and/or” refers to and encompasses any and all possible combinations of one or more of the associated listed items, as well as the lack of combinations when interpreted in the alternative (“or”).

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a complex comprises components A, B and C, it is specifically intended that any of A, B or C, or a combination thereof, can be omitted and disclaimed.

As used herein, the transitional phrase “consisting essentially of” (and grammatical variants) is to be interpreted as encompassing the recited materials or steps “and those that do not materially affect the basic and novel characteristic(s)” of the claimed invention. See, In re Herz, 537 F.2d 549, 551-52, 190 U.S.P.Q. 461, 463 (CCPA 1976) (emphasis in the original); see also MPEP § 2111.03. Thus, the term “consisting essentially of” as used herein should not be interpreted as equivalent to “comprising.”

The term “about,” as used herein when referring to a measurable value such as an amount or concentration and the like, is meant to encompass variations of ±10%, ±5%, ±1%, ±0.5%, or even ±0.1% of the specified value as well as the specified value. For example, “about X” where X is the measurable value, is meant to include X as well as variations of ±10%, ±5%, ±1%, ±0.5%, or even ±0.1% of X. A range provided herein for a measurable value may include any other range and/or individual value therein.

Recitation of ranges of values herein are merely intended to serve as a shorthand method of referring individually to each separate value falling within the range, unless otherwise indicated herein, and each separate value is incorporated into the specification as if it were individually recited herein. For example, if the range 10 to 15 is disclosed, then 11, 12, 13, and 14 are also disclosed.

As used herein, the terms “increase,” “increasing,” “enhance,” “enhancing,” “improve” and “improving” (and grammatical variations thereof) describe an elevation of about 1%, 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500% or more such as compared to another measurable property or quantity (e.g., a control value).

As used herein, the terms “reduce,” “reduced,” “reducing,” “reduction,” “diminish,” and “decrease” (and grammatical variations thereof), describe, for example, a decrease of about 1%, 5%, 10%, 15%, 20%, 25%, 30%, 35%, 40%, 45%, 50%, 55%, 60%, 65%, 70%, 75%, 80%, 85%, 90%, 95%, 97%, 98%, 99%, or 100% such as compared to another measurable property or quantity (e.g., a control value). In some embodiments, the reduction can result in no or essentially no (i.e., an insignificant amount, e.g., less than about 10% or even 5%) detectable activity or amount.

Like numbers refer to like elements throughout. In the figures, the thickness of certain lines, layers, components, elements or features may be exaggerated for clarity. The abbreviations “FIG. and “Fig.” for the word “Figure” can be used interchangeably in the text and figures.

It will be understood that when an element is referred to as being “on,” “attached” to, “connected” to, “coupled” with, “contacting,” etc., another element, it can be directly on, attached to, connected to, coupled with or contacting the other element or intervening elements may also be present. In contrast, when an element is referred to as being, for example, “directly on,” “directly attached” to, “directly connected” to, in “direct contact” with, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

It will be understood that, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present invention. The sequence of operations (or steps) is not limited to the order presented in the claims or figures unless specifically indicated otherwise.

As used herein, the term “solution” refers to a homogenous mixture of two or more substances wherein at least one gas, liquid, or solid substance (e.g., a solute) is dissolved into at least one liquid substance (e.g., a solvent). The solution may or may not be buffered so as to resist a change in the pH level when an acidic or basic component is added.

As used herein, the term “aqueous solution” refers to a solution in which the solvent is water. In some embodiments, the aqueous solution may be a saline solution (e.g., a solution of sodium chloride dissolved in water).

As used herein, the term “hydrogel” refers to a gel comprising water and a polymer network. Water may be the swelling agent for a hydrogel of the present invention. In some embodiments, a hydrogel of the present invention may contain water within the polymer network of the hydrogel, and the polymer network may be a three-dimensional (3D) polymer network. In some embodiments, a polymer network of a hydrogel of the present invention may comprise crosslinked polymers (e.g., crosslinked hydrophilic polymers). In some embodiments, a hydrogel of the present invention has a 3D structure in the presence of water and the polymer network may not dissolve in water. In some embodiments, a hydrogel of the present invention comprises a solution such that the solvent (e.g., water) and the solute (e.g., an orexin receptor ligand and/or a protease inhibitor) are distributed (e.g., uniformly distributed) within the hydrogel. A hydrogel of the present invention may be prepared to include (e.g., be loaded with) an agent (e.g., an orexin receptor ligand and/or a protease inhibitor) by contacting (e.g., soaking, submerging, and/or the like) a hydrogel and a composition comprising the agent, optionally wherein the agent is dissolved or suspended in the composition, thereby the agent is provided within the hydrogel. In some embodiments, an agent is present in an aqueous composition that is used to swell a polymer network (e.g., a modified alginate of the present invention) to thereby provide a hydrogel of the present invention comprising the agent. In some embodiments, a hydrogel of the present invention comprises an orexin receptor ligand and optionally a protease inhibitor. In some embodiments, a hydrogel of the present invention is devoid of a permeability enhancer and/or is devoid of a biologic that is not an orexin receptor ligand.

A hydrogel of the present invention may be formed as or in the form of a sheet, a bead, a capsule, a cube, a cylinder, and/or a sphere. In some embodiments, a hydrogel of the present invention is a microparticle (e.g., a microbead) or a nanoparticle. In some embodiments, a hydrogel of the present invention has at least one dimension (e.g., diameter, width, or length) in a range of about 100, 200, or 300 microns to about 400, 500, or 600 microns. A hydrogel of the present invention may have an elastic modulus in a range of about 1 or 5 kPa to about 10, 15, or 20 kPa. In some embodiments a hydrogel of the present invention has an elastic modulus of about 1, 2, 3, 4, 5, 6, 7, 8, 9, 10, 11, 12, 13, 14, 15, 16, 17, 18, 19, or 20 kPa.

A hydrogel of the present invention may be a cross-linked hydrogel (e.g., one or more polymer(s) of the polymer networks are crosslinked together). In some embodiments, a hydrogel of the present invention is cross-linked with a divalent cation (e.g., calcium). In some embodiments, a hydrogel of the present invention is cross-linked with about 25 mM to about 200 mM of the divalent cation (e.g., about 25, 30, 35, 40, 45, 50, 60, 70, 80, 90, 100, 110, 120, 130, 140, 150, 160, 170, 180, 190, or about 200 mM of the divalent cation). A divalent cation may be present in a hydrogel of the present invention in an amount of about 0.1, 0.2, 0.3, 0.4, or 0.5 mmol to about 0.6, 0.7, 0.8, 0.9, or 1 mmol. In some embodiments, a hydrogel of the present invention comprises an aqueous solution (e.g., a saline solution) that is optionally buffered. In some embodiments, an aqueous solution comprises one or more (e.g., 1, 2, 3, 4, 5, or more) different compound(s) that may be dissolved and/or suspended therein. For example, in some embodiments, an aqueous solution comprises an orexin receptor ligand and/or a protease inhibitor.

According to some embodiments, a hydrogel of the present invention comprises a modified carbohydrate (e.g., a modified alginate) that is optionally crosslinked. In some embodiments, a modified carbohydrate of the present invention is a modified carbohydrate as described in U.S. Pat. No. 10,766,970, the contents of which is incorporated herein by reference in its entirety. In some embodiments, a modified carbohydrate (e.g., a modified alginate) of the present invention comprises at least one unit having a structure of Formula I or Formula II:

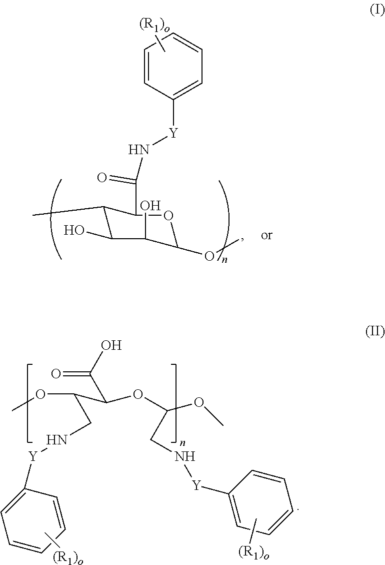

wherein
Y is absent or a C₁-C₄ alkyl or C₁-C₄ alkenyl;
R₁ is each independently selected from the group consisting of —H, —OH, —NH₂, —COOH, —C(O)OR′, —NO₂, —CN, —Br, —Cl, —F, —C₁-C₆ alkylhalide, unsubstituted or substituted —C₁-C₆ alkyl, unsubstituted or substituted —C₁-C₆ alkenyl, —SO₂H, —SO₃H, —COR′ (e.g., —COCH₃), —Si(OH)₃, —SO₂NH₂, —PO(OR′)₂, and —B(OH)₂, wherein R′ is unsubstituted or substituted alkyl, alkenyl, alkynyl, or aryl;
n is from 1 to 1,000,000; and
is 0, 1, 2, 3, 4, or 5.

In some embodiments, a modified carbohydrate (e.g., a modified alginate) of the present invention comprises at least one unit of Formula I that has at least one R₁ that is selected from the group consisting of —OH, —NH₂, —COOH, —C(O)OR′, —NO₂, —CN, —Br, —Cl, —F, —C₁-C₆ alkylhalide, unsubstituted or substituted —C₁-C₆ alkyl, unsubstituted or substituted —C₁-C₆ alkenyl, —SO₂H, —SO₃H, —COR′ (e.g., —COCH₃), —Si(OH)₃, —SO₂NH₂, —PO(OR′)₂, and —B(OH)₂, wherein each R′ is independently selected from the group consisting of unsubstituted or substituted alkyl, alkenyl, alkynyl and aryl. In some embodiments, a modified carbohydrate (e.g., a modified alginate) of the present invention comprises at least one unit of Formula I that has at least one R′ that is hydrogen. In some embodiments, a modified carbohydrate (e.g., modified alginate) of the present invention comprises at least one unit of Formula I that has at least one R₁ in the para position and is selected from the group consisting of —OH, —NH₂, —COOH, —C(O)OR′, —NO₂, —CN, —Br, —Cl, —F, —C₁-C₆ alkylhalide, unsubstituted or substituted-C₁-C₆ alkyl, unsubstituted or substituted —C₁-C₆ alkenyl, —SO₂H, —SO₃H, —COR′ (e.g., —COCH₃), —Si(OH)₃, —SO₂NH₂, —PO(OR′)₂, and —B(OH)₂, and wherein R′ is unsubstituted or substituted alkyl, alkenyl, alkynyl, or aryl. In some embodiments, a modified carbohydrate (e.g., modified alginate) of the present invention comprises at least one unit of Formula I that has at least one R₁ in the para position and is selected from the group consisting of —NH₂, —COOH, —C(O)OR′, —NO₂, —CN, —Br, —Cl, —F, —C₁-C₆ alkylhalide, unsubstituted or substituted —C₁-C₆ alkyl, unsubstituted or substituted —C₁-C₆ alkenyl, —SO₂H, —SO₃H, —COR′ (e.g., —COCH₃), —Si(OH)₃, —SO₂NH₂, —PO(OR′)₂, and —B(OH)₂, and wherein R′ is unsubstituted or substituted alkyl, alkenyl, alkynyl, or aryl.

In some embodiments, a modified carbohydrate (e.g., a modified alginate) of the present invention comprises at least one unit of Formula II that has at least one R₁ that is selected from the group consisting of —OH, —NH₂, —COOH, —C(O)OR′, —NO₂, —CN, —Br, —Cl, —F, —C₁-C₆ alkylhalide, unsubstituted or substituted —C₁-C₆ alkyl, unsubstituted or substituted —C₁-C₆ alkenyl, —SO₂H, —SO₃H, —COR′ (e.g., —COCH₃), —Si(OH)₃, —SO₂NH₂, —PO(OR′)₂, and —B(OH)₂, wherein each R′ is independently selected from the group consisting of unsubstituted or substituted alkyl, alkenyl, alkynyl and aryl. In some embodiments, a modified carbohydrate (e.g., a modified alginate) of the present invention comprises at least one unit of Formula II that has at least one R₁ that is hydrogen. In some embodiments, a modified carbohydrate (e.g., modified alginate) of the present invention comprises at least one unit of Formula II that has at least one R₁ in the para position and is selected from the group consisting of —OH, —NH₂, —COOH, —C(O)OR′, —NO₂, —CN, —Br, —Cl, —F, —C₁-C₆ alkylhalide, unsubstituted or substituted-C₁-C₆ alkyl, unsubstituted or substituted —C₁-C₆ alkenyl, —SO₂H, —SO₃H, —COR′ (e.g., —COCH₃), —Si(OH)₃, —SO₂NH₂, —PO(OR′)₂, and —B(OH)₂, and wherein R′ is unsubstituted or substituted alkyl, alkenyl, alkynyl, or aryl. In some embodiments, a modified carbohydrate (e.g., modified alginate) of the present invention comprises at least one unit of Formula II that has at least one R₁ in the para position and is selected from the group consisting of —NH₂, —COOH, —C(O)OR′, —NO₂, —CN, —Br, —Cl, —F, —C₁-C₆ alkylhalide, unsubstituted or substituted —C₁-C₆ alkyl, unsubstituted or substituted —C₁-C₆ alkenyl, —SO₂H, —SO₃H, —COR′ (e.g., —COCH₃), —Si(OH)₃, —SO₂NH₂, —PO(OR′)₂, and —B(OH)₂, and wherein R′ is unsubstituted or substituted alkyl, alkenyl, alkynyl, or aryl.

A modified carbohydrate (e.g., a modified alginate) of the present invention may comprise at least one unit of Formula I and/or at least one unit of Formula II. In some embodiments, one or more functional groups in a modified carbohydrate (e.g., a modified alginate) of the present invention comprising at least one unit of Formula I and/or at least one unit of Formula II may be protonated or deprotonated, optionally one or more R₁ in the modified carbohydrate (e.g., a modified alginate) may be protonated or deprotonated. In some embodiments, the protonation state (i.e., protonated or deprotonated) of one or more functional groups in a modified carbohydrate (e.g., a modified alginate) of the present invention may depend on the pH of the environment that the modified carbohydrate (e.g., a modified alginate) is exposed to and/or in contact with.

For simplicity, described herein are further embodiments with respect to certain carbohydrates, such as modified alginate, but it should be understood that other carbohydrates may be used.

A hydrogel of the present invention may comprise a modified alginate. A modified alginate of the present invention may be a chemically modified alginate. In some embodiments, a hydrogel of the present invention comprises an alginate that has been oxidized and/or modified (e.g., modified with a reactant to attach a moiety (e.g., a modification moiety) to the alginate). In some embodiments, about 0.1%, 0.5%, 1%, or 2% to about 5%, 10%, or 15% of the units (e.g., polysaccharide units) of a modified alginate may be modified (e.g., include a functional group (e.g., modification moiety) that is not naturally present in the alginate). In some embodiments, about 0.1%, 0.5%, 1%, 2%, 3%, 4%, 5%, 6%, 7%, 8%, 9%, 10%, 11%, 12%, 13%, 14%, or 15% of the units (e.g., polysaccharide units) of a modified alginate may be modified. In some embodiments, a modified alginate of the present invention is present in a hydrogel of the present invention in an amount of about 0.1%, 0.5%, 1%, or 2% to about 3%, 4%, 5%, 6%, 7%, 8%, 9%, or 10% w/v of the hydrogel.

In some embodiments, a modified alginate includes a moiety that has a pKa in a range of about 3, 3.5, or 4 to about 4.5, 5, 5.5, or 6. The modified alginate may have been modified to include the moiety having a pKa in a range of about 3, 3.5, or 4 to about 4.5, 5, 5.5, or 6. In some embodiments, a modified alginate includes a moiety that has a pKa of about 3, 3.5, 4, 4.5, 5, 5.5, or 6. In some embodiments, a modified alginate includes at least one unit of Formula I and/or Formula II that includes at least one R₁ that has a pKa in a range of about 3, 3.5, or 4 to about 4.5, 5, 5.5, or 6. In some embodiments, a modified alginate includes at least one unit of Formula I and/or Formula II that includes at least one R₁ that has a pKa of about 3, 3.5, 4, 4.5, 5, 5.5, or 6.

Alginate used to prepare a modified alginate of the present invention may be of any type. Alginate is a polysaccharide composed of randomly oriented blocks of monomers of (1-4)-linked β-D-mannuronic acid (M) and α-L-guluronic acid (G). In some embodiments, a modified alginate of the present invention may be prepared and/or formed using an alginate having about 10%, 20%, 30%, 40%, 50%, 60%, 70%, 80%, or 90% of the total number of polysaccharide units be M units or G units. In some embodiments, an alginate used to prepare a modified alginate of the present invention may be a natural and/or unmodified alginate. In some embodiments, an alginate that is used to prepare a modified alginate of the present invention may be an oxidized alginate (e.g., alginate oxidized with an oxidizing agent (e.g., sodium periodate)). In some embodiments, an oxidized alginate may be prepared by oxidizing a vicinal diol in the alginate chain to an aldehyde.

A modified alginate of the present invention may be prepared by reacting an alginate with a reactant that is selected from the group consisting of 4-(2-aminoethyl)benzoic acid, 4-(2-aminomethyl)benzoic acid, 4-(2-aminoethyl)aniline, (2-ethylamino) 4-methyl benzene, 4-(2-aminoacetyl)-benzoic acid, 4-(2-aminoethyl)salicylic acid, methyl 4-(2-aminoethyl)benzoate, phenethylamine, 1-[4-(2-aminoethyl)phenyl]ethanone HCl, and/or 4-(2-aminomethyl)aniline and/or esters thereof. In some embodiments, a modified alginate of the present invention is a 2-amino ethyl aromatic (e.g., a 2-amino ethyl benzyl) modified alginate. In some embodiments, a modified alginate of the present invention is a 4-(2-aminoethyl)benzoic acid modified alginate (e.g., an alginate (optionally an oxidized alginate) that is modified (e.g., reacted) with 4-(2-aminoethyl)benzoic acid). In some embodiments, a modified alginate of the present invention is an aminomethyl aromatic (e.g., a 2-aminomethyl benzyl) modified alginate.

A reactant may be incorporated into an alginate in an amount of about 1% to about 20% based on the average number of polysaccharide units in the alginate to provide a modified alginate. In some embodiments, a reactant is incorporated into an alginate in an amount of about 1%, 2%, 3%, 4%, 5%, 6%, 7%, 8%, 9%, 10%, 11%, 12%, 13%, 14%, 15%, 16%, 17%, 18%, 19%, or 20% based on the average number of polysaccharide units in the alginate. In some embodiments, about 90%, 91%, 92%, 93%, 94%, 95%, 96%, 97%, 98%, 99%, or 100% of a reactant is chemically bound (e.g., covalently bound) to an alginate to provide the modified alginate, rather than free or trapped within the polymer. In some embodiments, a reactant may be reacted with an oxidized alginate (e.g., alginate in which a vicinal diol in the alginate chain is oxidized to an aldehyde) to provide the modified alginate.

In some embodiments, a modified alginate and/or hydrogel of the present invention may be stable (e.g., may not degrade and/or may not dissolve in) in an acidic environment (e.g., an acidic composition; e.g., a composition with a pH less than 7.0 (e.g., a pH of 1.0, 1.5, 2, 2.5, 3, 3.5, 4, 4.5, 5, 5.5, 6, 6.5, or 6.9)) such as an acidic environment similar to the stomach environment (e.g., the stomach environment of a subject). In some embodiments, a modified alginate and/or hydrogel of the present invention may disintegrate, degrade, and/or dissolve in a basic environment (e.g., an environment comprising a composition with a pH greater than 7.0 (e.g., a pH of 7.1, 7.5, 8, 8.5, 9, 9.5, 10, 10.5, 11, 11.5, 12, 12.5, 13, 13.5, or 14) such as a basic environment similar to the small intestine environment (e.g., the small intestine environment of a subject). In some embodiments, a hydrogel and/or modified alginate of the present invention may degrade in about 5, 10, 15, 20, 25, or 30 minutes to about 40, 60, 70, 80, 90, 100, 110, 120, 130, 140, or 150 minutes following exposure to (e.g., contact with) a composition (e.g., an aqueous composition) having a pH of above 7 (e.g., about 7.5 or more) and/or following administration to a subject and reaching the intestinal tract (e.g., the small intestine).

According to some embodiments of the present invention provided is a composition comprising a hydrogel of the present invention. In some embodiments, a hydrogel of the present invention may provide a barrier to a bioactive substance (e.g., a medicine, drug, enzyme, protein, nucleic acid, peptide, hormone, vaccine, vitamin, mineral, and/or micronutrient). The hydrogel may coat at least a portion of the bioactive substance and/or encapsulate at least a portion of the bioactive substance. In some embodiments, a hydrogel of the present invention comprises a bioactive substance and the hydrogel may prevent and/or reduce degradation and/or oxidation of the bioactive substance. In some embodiments, a modified alginate of the present invention is present in a hydrogel of the present invention in an amount of about 0.1%, 0.5%, 1%, or 2% to about 3%, 4%, 5%, 6%, 7%, 8%, 9%, or 10% w/v of the hydrogel.

In some embodiments, a composition and/or a hydrogel of the present invention comprises an orexin receptor ligand and/or a protease inhibitor. In some embodiments, a composition and/or hydrogel of the present invention comprises an orexin receptor ligand and is devoid of a protease inhibitor. The hydrogel may coat and/or encapsulate at least a portion (e.g., about 50%, 60%, 70%, 80%, 90%, or 100%) of the orexin receptor ligand and/or protease inhibitor. In some embodiments, a hydrogel of the present invention comprises an orexin receptor ligand and/or protease inhibitor that is uniformly distributed within the hydrogel.

According to some embodiments, a composition and/or hydrogel of the present invention may increase the shelf life of an orexin receptor ligand and/or a protease inhibitor when present in a hydrogel of the present invention. For example, the shelf life of the orexin receptor ligand and/or the protease inhibitor in liquid or powder form may be increased when present in a hydrogel of the present invention. “Shelf life” as used herein refers to the length of time a substance maintains a given level of activity in an unopened package stored under recommended storage conditions. The shelf life may, for example, be evidenced by the “use by” or “best if used by” date for the product and/or the manufacturer's expiration date of the product (i.e., the “predicted shelf life”) and/or the actual product characteristics after the specified period of time (i.e., the “actual shelf life”). Accordingly, the term “shelf life” as used herein should be construed as including both the actual shelf life of the product and the predicted shelf life of the product unless stated otherwise. In some embodiments, shelf life may be determined by extrapolation of data at accelerated temperatures, such as, for example, by using the Arrhenius equation. In some embodiments, shelf life may be determined using linear regression analysis, such as, for example, when the kinetics of the orexin receptor ligand and/or the protease inhibitor degradation is not temperature dependent. In some embodiments, shelf life may be evaluated and/or determined by measuring the orexin receptor ligand and/or the protease inhibitor, such as, for example, using high pressure liquid chromatography. In some embodiments, the shelf life of a composition of the present invention that includes a hydrogel comprising an orexin receptor ligand and/or a protease inhibitor is the time that the orexin receptor ligand and/or the protease inhibitor in the hydrogel maintains at least 50% (e.g., about 55%, 60%, 65%, 70%, 75%, 80%, 85%, 90%, 95%, or more) of a given activity (e.g., in the case of the orexin receptor ligand, the ability to bind to and affect the function of (e.g., activate, increase activity, decrease activity, antagonize binding of a separate ligand, and the like) an orexin receptor; in the case of a protease inhibitor, reduce the activity of at least one target protease) compared to the initial activity of the orexin receptor ligand and/or the protease inhibitor prior to incorporation into the hydrogel.

As used herein, the term “orexin receptor ligand” refers to a compound that partially or fully binds to an orexin receptor protein. In some embodiments, an orexin receptor ligand specifically binds to an orexin receptor protein. In some embodiments, the orexin receptor protein is a G protein-coupled receptor (GPCR) such as orexin receptor type 1 (OX1R) and/or orexin receptor type 2 (OX2R). In some embodiments, the orexin receptor ligand may increase the activity of the orexin receptor protein. In some embodiments, the orexin receptor ligand may decrease the activity of the orexin receptor protein. An orexin receptor ligand of the present invention may be an orexin peptide (e.g., orexin-A and/or orexin-B) or an orexin receptor antagonist (also referred to as an orexin antagonist). In some embodiments, the orexin antagonist is almorexant. An orexin receptor ligand may be present in a hydrogel of the present invention in a concentration of about 1, 5, 10, 25, 50, 75, 100, 150, or 200 μg of the orexin receptor ligand per mL of the hydrogel to about 250, 300, 350, 400, 450, or 500 μg of the orexin receptor ligand per mL of the hydrogel. In some embodiments, an orexin receptor ligand may be present in a hydrogel of the present invention in a concentration of about 1, 5, 10, 25, 50, 75, 100, 150, 200, 250, 300, 350, 400, 450, or 500 μg of the orexin receptor ligand per mL of the hydrogel.

A composition of the present invention may further comprise a therapeutic agent. In some embodiments, the therapeutic agent may be a biologic, a chemotherapeutic, and/or an immunotherapeutic. In some embodiments, a hydrogel of the present invention comprising an orexin receptor ligand and/or protease inhibitor may improve the therapeutic activity of a therapeutic agent (e.g., a biologic, chemotherapeutic, and/or immunotherapeutic). In some embodiments, a chemotherapeutic useful for this invention is paclitaxel. One or more (e.g., 1, 2, 3, 4, or more) therapeutic agent(s) may be present in a hydrogel of the present invention. A therapeutic agent may be present in a hydrogel of the present invention in a concentration of about 1, 5, 50, 100, 250, or 500 ng of the therapeutic agent per mL of the hydrogel to about 0.5, 1, 2, 4, 6, 8, or 10 μg of the therapeutic agent per mL of the hydrogel. In some embodiments, a therapeutic agent is present in a hydrogel of the present invention in a total concentration of about 1 or 5 unit(s) to about 10, 15, or 20 units.

As used herein, the term “biologic” refers to a polypeptide (e.g., a protein), an enzyme, a hormone, a nucleotide, and/or a nucleic acid (e.g., a polynucleotide). In some embodiments, a biologic is a compound that was manufactured in, extracted from, or semisynthesized from a living cell, tissue, or organism. In some embodiments, a biologic is synthetically obtained (e.g., produced). In some embodiments, a biologic that is present in a composition and/or hydrogel of the present invention is not an orexin receptor ligand.

As used herein, the term “protease inhibitor” describes a compound that partially or fully inhibits or blocks the activity of an enzyme that breaks apart a protein (e.g., cleaves one or more polypeptide bonds of the protein). One or more (e.g., 1, 2, 3, 4, or more) protease inhibitor(s) may be present in a hydrogel of the present invention. Exemplary protease inhibitors include, but are not limited to, Bowman-Birk inhibitors, Kunitz-type inhibitors, α-amylase inhibitors, and/or trypsin inhibitors. In some embodiments, a hydrogel of the present invention comprises aprotinin. In some embodiments, a protease inhibitor of the present invention has a molecular weight of about 500 or 1000 Daltons to about 2000, 3000, 4000, 5000, 6000, or 7000 Daltons. In some embodiments, a protease inhibitor is present in a hydrogel of the present invention in a concentration of about 0.01, 0.05, or 0.1 mg of the protease inhibitor per mL of the hydrogel to about 0.5, 1, 2, 3, 4, or 5 mg of the protease inhibitor per mL of the hydrogel.

In some embodiments, a hydrogel of the present invention is suspended in a carrier, optionally a pharmaceutical carrier. In some embodiments, a hydrogel is suspended in water, an aqueous solution, a suppository base, and/or an oil. In some embodiments, a hydrogel is suspended in a carrier in which the hydrogel and/or its polymer network does not dissolve and/or degrade. As used herein, the term “pharmaceutical carrier” describes any pharmaceutically acceptable solvent, diluent, liposome, or other excipient that aids in the delivery of a composition to an organism (e.g., an animal such as a human) and/or to a target tissue. In some embodiments, a pharmaceutical carrier is a liquid, a solid, or a semi-solid. The selection of a specific pharmaceutical carrier may depend on the desired features of the composition in which it is included, such as a need to enhance the delivery of a composition to a target tissue type, the required stability of the composition, a need to control the release schedule of the composition within the animal (e.g., a human), and/or the administration method to the animal (e.g., a human) (e.g., orally administered capsules, orally administered liquids or gels, rectally administered suppositories, injectable solutions, or other administration methods).

In some embodiments, a hydrogel and/or composition of the present invention is provided in a capsule (e.g., a biocompatible capsule). In some embodiments, a hydrogel and/or composition of the present invention are contained within a coating that optionally is configured for oral delivery and/or provides the hydrogel and/or composition in a form for oral delivery. In some embodiments, a capsule and/or coating comprises a cellulose. In some embodiments, a hydrogel of the present invention may be sized and/or configured for a subject to swallow with or without water.

In some embodiments, a hydrogel and/or composition of the present invention is provided in a suppository. In some embodiments, a hydrogel and/or composition of the present invention are contained within a suppository base that is configured for rectal delivery and/or provides the hydrogel and/or composition in a form for rectal delivery. In some embodiments, the suppository base may be an FDA-approved suppository material.

In some embodiments, a hydrogel and/or composition of the present invention is provided in a pharmaceutical carrier that is configured for intravenous injection, intramuscular injection, intratumoral injection, depot injection, intraperitoneal injection, intracerebroventricular injection, or subcutaneous injection. In some embodiments, the pharmaceutical carrier may be saline, optionally phosphate-buffered saline.

A composition of the present invention may include one or more (e.g., 1, 2, 3, 4, 5, or more) hydrogel(s) of the present invention, each of the one or more hydrogel(s) may be discrete and/or separate from another hydrogel present in the composition, and the composition may optionally include a carrier. In some embodiments, a composition of the present invention comprises a plurality of hydrogels of the present invention that are the same. In some embodiments, a composition of the present invention includes two or more different hydrogels with each of the two or more different hydrogels including a different agent. For example, in some embodiments, a composition of the present invention comprises a first hydrogel comprising an orexin receptor ligand and a second hydrogel comprising a protease inhibitor, optionally wherein the first and second hydrogels are configured to release the orexin receptor ligand and protease inhibitor the same or differently (e.g., the first hydrogel comprises a modified alginate hydrogel that has a different concentration of polysaccharide units modified than the second hydrogel that comprises a modified alginate hydrogel). In some embodiments, a composition of the present invention comprises a first hydrogel comprising an orexin receptor ligand and a second hydrogel comprising the orexin receptor ligand, optionally wherein the first and second hydrogels are configured to release the orexin receptor ligand differently (e.g., the first hydrogel comprises a modified alginate hydrogel that has a different concentration of polysaccharide units modified than the second hydrogel that comprises a modified alginate hydrogel). In some embodiments, differently releasing the protease inhibitor and the orexin receptor ligand comprises releasing the protease inhibitor prior to releasing the orexin receptor ligand. When two or more different hydrogels are present in a composition of the present invention, the hydrogels may comprise the same polymer network (e.g., modified alginate) or a different polymer network. In some embodiments, a composition of the present invention comprises two different hydrogels that have the same polymer network (e.g., modified alginate). In some embodiments, a composition of the present invention comprises two different hydrogels that have the same polymer network (e.g., modified alginate), but are modified differently (e.g., modified with different reactants) and/or have a different level of modification (e.g., have a different amount of modified polysaccharide units). In some embodiments, a composition of the present invention comprises two different hydrogels that each comprise a modified alginate that is modified with the same reactant, but one of the two hydrogels comprises a greater number of modified polysaccharide units. In some embodiments, a composition of the present invention comprises a hydrogel that comprises a modified alginate of the present invention in which about 1% of the polysaccharide units of the modified alginate are modified. In some embodiments, a composition of the present invention comprises a hydrogel that comprises a modified alginate of the present invention in which about 2% of the polysaccharide units of the modified alginate are modified.

In some embodiments, a composition of the present invention comprises a first hydrogel comprising an orexin receptor ligand and/or a second hydrogel comprising a protease inhibitor. An orexin receptor ligand may be present in a composition of the present invention in a total concentration of about 0.1 μM to about 10 μM (e.g., about 0.1 μM to about 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, or 10 μM). A protease inhibitor (e.g., aprotinin) may be present in a composition of the present invention in a total concentration of about 50 or 100 mg of the protease inhibitor per mL of the composition to about 150 or 200 mg of the protease inhibitor per mL of the composition.

In some embodiments, a composition of the present invention comprises a first hydrogel comprising a first agent and a second hydrogel including a second agent, wherein the first and second agents are different. A composition of the present invention comprising two or more hydrogels that each include a different agent may include each of the hydrogels in the same amount or in different amounts. In some embodiments, a composition of the present invention has a ratio of hydrogel particulates (e.g., hydrogel particles such as beads) in a range of about 1:1 to about 1:2, 1:3, 1:4, or 1:5 or about 1:1 to about 3:2 (first hydrogel particulates:second hydrogel particulates). In some embodiments, the amount of second hydrogel particulates in a composition of the present invention is a percentage of an amount of first hydrogel particulates (e.g., about 10%, 20%, 30%, 40%, 50%, 60%, 70%, 80%, 90%, 100%, 110%, 120%, 130%, 140%, 150%, 160%, 170%, 180%, 190%, or about 200%).

A hydrogel and/or modified alginate of the present invention may coat and/or encapsulate at least a portion of a compound (e.g., an orexin receptor ligand and/or protease inhibitor), may protect the compound at pH levels that are found in the stomach (e.g., acidic pH of about 1 to about 3), and/or may allow and/or provide for the compound to be made available at pH levels that are found in the intestines (e.g., basic pH of about 7 to about 9) as the alkaline environment may allow for the hydrogel and/or modified alginate to be broken down, which may allow for access to and/or release of the compound. In some embodiments, a composition and/or hydrogel of the present invention reduces or prevents exposure of a compound (e.g., an orexin receptor ligand and/or protease inhibitor) to an acidic environment (e.g., to the stomach environment), which may increase and/or improve activity and/or bioavailability of the compound, optionally upon administration (e.g., oral administration) to a subject.

In some embodiments, a hydrogel and/or modified alginate of the present invention may coat and/or encapsulate at least a portion of a compound (e.g., an orexin receptor ligand and/or protease inhibitor) and/or may allow and/or provide for the compound to be made available at pH levels that are found in the stomach (e.g., pH of about 1 to about 3). In some embodiments, a composition of the present invention comprises a hydrogel of the present invention and a carrier (e.g., saline) having a pH of about 7 to about 8 (e.g., about 7.5). In some embodiments, a carrier of the present invention may have a pH suitable and/or configured for releasing the contents of a hydrogel (e.g., an orexin receptor ligand and/or protease inhibitor) at a desired time and/or location optionally upon administration to a subject. In some embodiments, a hydrogel and/or modified alginate of the present invention is configured to release its contents (e.g., an orexin receptor ligand and/or protease inhibitor) at a site (e.g., an administration and/or injection site) in a subject (e.g., in a vein, blood, muscle, tumor, peritoneum, subcutis, rectum, and/or vaginal cavity of a subject).

In some embodiments, a hydrogel and/or a modified alginate of the present invention may be able to withstand the pH of saliva (generally a pH of about 6.5 to about 7.4). In some embodiments, a hydrogel and/or modified alginate may be able to withstand the pH of saliva for a sufficient amount of time such that, upon oral administration, the hydrogel may be able to reach the stomach and/or intestine (e.g., small intestine) with at least a portion of the compound (e.g., about 20%, 30%, 40%, 50%, 55%, 60%, 65%, 70%, 75%, 80%, 85%, 90%, 95%, or 100%) still encapsulated and/or coated. In some embodiments, a hydrogel and/or modified alginate of the present invention is stable for at least about 5, 10, or 15 minutes at a pH above 7. In some embodiments, upon exposure to the pH of the small intestine, a compound (e.g., an orexin receptor ligand and/or protease inhibitor) present in a hydrogel may become available for its intended benefit (e.g., is released from the hydrogel). In some embodiments, upon exposure to the pH of the stomach, a compound (e.g., an orexin receptor ligand and/or protease inhibitor) present in a hydrogel may become available for its intended benefit (e.g., is released from the hydrogel). In some embodiments, upon exposure in a subject, a compound (e.g., an orexin receptor ligand and/or protease inhibitor) present in a hydrogel may become available for its intended benefit (e.g., is released from the hydrogel).

According to some embodiments, provided is a method of treating a subject such as a subject in need thereof, the method comprising administering a hydrogel and/or a composition of the present invention to the subject. In some embodiments, a therapeutically effective amount of a hydrogel of the present invention and/or a therapeutically effective amount of a compound (e.g., an orexin receptor ligand and/or protease inhibitor) that is present in a hydrogel of the present invention is administered to a subject. As used herein, the term “therapeutically effective amount” refers to an amount of a compound (e.g., an orexin receptor ligand, protease inhibitor, and/or hydrogel) that elicits a therapeutically useful response in a subject. Those skilled in the art will appreciate that the therapeutic effects need not be complete or curative, as long as some benefit is provided to the subject. In some embodiments, a therapeutically effective amount of a hydrogel of the present invention may include delivering a therapeutically effective amount of an agent such as an orexin receptor ligand (e.g., an orexin peptide (e.g., orexin-A and/or orexin-B) or an orexin receptor antagonist) present in the hydrogel. In some embodiments, eliciting a useful response in a subject comprises administering a first hydrogel comprising an orexin receptor ligand, and optionally a second hydrogel comprising a protease inhibitor. In some embodiments, a method of the present invention comprises administering a therapeutically effective amount of a first hydrogel comprising an orexin receptor ligand and optionally a therapeutically effective amount of a second hydrogel comprising a protease inhibitor.

“Treat,” “treating” or “treatment of” (and grammatical variations thereof) as used herein refer to any type of treatment that imparts a benefit to a subject and may mean that the severity of the subject's disease, disorder, or condition is reduced, at least partially improved or ameliorated, and/or that some alleviation, mitigation or decrease in at least one clinical symptom associated with a disease, disorder, or condition is achieved and/or there is a delay in the progression of a symptom. In some embodiments, the severity of a symptom associated with a subject's disease, disorder, or condition may be reduced compared to the severity of the symptom in the absence of a method of the present invention.

In some embodiments, a hydrogel and/or an agent present therein (e.g., an orexin receptor ligand and/or protease inhibitor) may be administered in a treatment effective amount. A “treatment effective” amount as used herein is an amount that is sufficient to treat (as defined herein) a subject. Those skilled in the art will appreciate that the therapeutic effects need not be complete or curative, as long as some benefit is provided to the subject. In some embodiments, a treatment effective amount may be achieved by administering a composition of the present invention.

The present invention finds use in both veterinary and medical applications. Subjects suitable to be treated with a method of the present invention include, but are not limited to, mammalian subjects. Mammals of the present invention include, but are not limited to, canines, felines, bovines, caprines, equines, ovines, porcines, rodents (e.g., rats and mice), lagomorphs, primates (e.g., simians and humans), non-human primates (e.g., monkeys, baboons, chimpanzees, gorillas), and the like, and mammals in utero. Any mammalian subject in need of being treated according to the present invention is suitable. Human subjects of both genders and at any stage of development (i.e., neonate, infant, juvenile, adolescent, adult) may be treated according to the present invention. In some embodiments of the present invention, the subject is a mammal and in certain embodiments the subject is a human. Human subjects include both males and females of all ages including fetal, neonatal, infant, juvenile, adolescent, adult, and geriatric subjects as well as pregnant subjects. In particular embodiments of the present invention, the subject is a human adolescent and/or adult.

A method of the present invention may also be carried out on animal subjects, particularly mammalian subjects such as mice, rats, dogs, cats, livestock and horses for veterinary purposes, and/or for drug screening and drug development purposes.

In some embodiments, a subject is “in need of” or “in need thereof” a method of the present invention, for example, the subject has findings typically associated with a disease, disorder, or condition (e.g., cancer), is suspected to have a disease, disorder, or condition, and/or the subject has a disease, disorder, or condition. In some embodiments, a subject has cancer (e.g., colorectal cancer, pancreatic cancer, liver cancer, stomach cancer, cervical cancer, brain cancer, adrenal cancer, and/or prostate cancer). In some embodiments, a method of the present invention may treat cancer in a subject. In some embodiments, a subject has a sleep disorder (e.g., insomnia). In some embodiments, a method of the present invention may treat a sleep disorder in a subject. In some embodiments, a subject has a feeding disorder and/or an eating disorder. In some embodiments, a method of the present invention may treat a feeding disorder and/or eating disorder in a subject. In some embodiments, a subject has a substance abuse disorder (e.g., an addiction). In some embodiments, a method of the present invention may treat a substance abuse disorder in a subject. In some embodiments, a subject has neuropathic pain. In some embodiments, a method of the present invention may treat neuropathic pain in a subject.

A method of the present invention may comprise administering a single composition comprising one or more hydrogel(s) of the present invention that may be different from each other and/or administering two or more different compositions of the present invention. In some embodiments, a method of the present invention comprises administering a first composition comprising a first hydrogel comprising an orexin receptor ligand to a subject. In some embodiments, a method of the present invention is devoid of administering a protease inhibitor. In some embodiments, a method of the present invention comprises administering a first composition comprising a first hydrogel comprising an orexin receptor ligand to a subject and administering a protease inhibitor to the subject, optionally wherein the protease inhibitor is present in the same composition and/or hydrogel and/or in a different composition and/or hydrogel as the orexin receptor ligand. In some embodiments, the protease inhibitor is present in a second hydrogel that is separate from the first hydrogel comprising the orexin receptor ligand. In some embodiments, the protease inhibitor is present in a second composition that is separate from the first composition comprising the orexin receptor ligand, but the two compositions may be administered together or the two compositions may be administered separately. In some embodiments, the administering comprises separately administering the first hydrogel and the second hydrogel to the subject at different times. In some embodiments, the administering comprises concurrently administering the first hydrogel and the second hydrogel to the subject. In some embodiments, the administering comprises concurrently administering the first hydrogel and the second hydrogel to the subject, optionally wherein the first hydrogel and the second hydrogel are present in the same composition.

A method and/or composition of the present invention may provide for a sequential release of two different agents such as an orexin receptor ligand and a protease inhibitor. In some embodiments, a hydrogel and/or modified alginate of the present invention may be configured to release an agent at a particular time. In some embodiments, a method and/or composition of the present invention comprises a hydrogel comprising a protease inhibitor that, following administration to a subject, releases the protease inhibitor prior to a hydrogel comprising an orexin receptor ligand that is administered to the subject optionally when administered at the same time as the hydrogel comprising the protease inhibitor. In some embodiments, a composition of the present invention comprises at least one hydrogel that releases its contents and/or degrades at a different rate than another hydrogel present in the same composition or in a different composition optionally administered at the same time as the at least one hydrogel. In some embodiments, a method and/or composition of the present invention may provide for a sequential release of two different agents such as a protease inhibitor and an orexin receptor ligand. In some embodiments, a method of the present invention comprises administering to a subject a composition comprising two different hydrogels that each release an agent concurrently.

A method of the present invention may improve bioavailability of an orexin receptor ligand. For example, a method of the present invention may comprise orally administering to a subject a composition comprising a hydrogel that includes an orexin receptor ligand, wherein the hydrogel may prevent the release of the orexin receptor ligand in the stomach, where it could be degraded due to the pH level in the stomach, and releases insulin in the small intestine.

In some embodiments, a method of the present invention comprises orally administering a composition of the present invention. Formulations suitable for oral administration may be presented in discrete units, such as capsules, cachets, lozenges, or tablets, each containing a predetermined amount of an agent (e.g., an orexin receptor ligand) and/or hydrogel; as a solution or a suspension in an aqueous or non-aqueous liquid; or as an oil-in-water or water-in-oil emulsion. Such formulations may be prepared by any suitable method of pharmacy which includes the step of bringing into association a hydrogel of the present invention and a suitable carrier (which may contain one or more accessory ingredients). In some embodiments, a composition of the present invention may be prepared by uniformly admixing a hydrogel of the present invention with a liquid carrier.

In some embodiments, a method of the present invention comprises rectally or vaginally administering a composition of the present invention optionally as a suppository. Formulations suitable for rectal and/or vaginal administration as a suppository may include a suppository base containing a predetermined amount of an agent (e.g., an orexin receptor ligand) and/or hydrogel of the present invention. In some embodiments, a composition of the present invention may be prepared by uniformly admixing a hydrogel of the present invention with a suppository base. In some embodiments, a hydrogel of the present invention may be in the form of a suppository and optionally may be devoid of a suppository base.

In some embodiments, a method of the present invention comprises administering a composition of the present invention by injection. In some embodiments, the injection may be an intravenous injection, intramuscular injection, intratumoral injection, depot injection, intraperitoneal injection, intracerebroventricular injection, or subcutaneous injection. Formulations suitable for an injection may include a pharmaceutical carrier containing a predetermined amount of an agent (e.g., an orexin receptor ligand) and/or hydrogel optionally in a solution or a suspension including an aqueous liquid or in an oil-in-water or water-in-oil emulsion.

A method of the present invention may comprise administering an orexin receptor ligand to a subject for a period of time so as to produce a therapeutically useful response over a time period. In some embodiments, the therapeutically useful response may be provided for a time period up to the time when an immediately subsequent dose of the orexin receptor ligand is administered to the subject. In some embodiments, a method of the present invention comprises administering an orexin receptor ligand and/or protease inhibitor that is structurally intact at the time of release from a hydrogel in which it is present and/or that has an activity that is within #20% of its original activity prior to incorporation in the hydrogel. In some embodiments, following administering to a subject a composition of the present invention comprising an orexin receptor ligand, the peak concentration of the orexin receptor ligand is reached within 4 hours after administration of the composition.

In some embodiments, a method of the present invention provides a continuous release of a protease inhibitor and/or an orexin receptor ligand for a period of time. In some embodiments, the period of time of continuous release is about 1, 2, 4, 6, 8, 10, 12, 14, 16, 18, 20, 22, or 24 hour(s); or about 1, 2, 3, 4, 5, 6, 7, 8, 9, 10, 11, or 12 day(s) or week(s) or more. In some embodiments, about 20% to about 100% (e.g., about 20%, 30%, 40%, 50%, 55%, 60%, 65%, 70%, 75%, 80%, 85%, 90%, 95%, or 100%) of the protease inhibitor and/or the orexin receptor ligand present in a hydrogel is released from the hydrogel within about 1 day to about 3 months (e.g., about 1, 5, 10, 15, 20, 25, 30, 35, 40, 45, 50, 55, 60, 65, 70, 75, 80, 85, or 90 days) after administration to a subject. In some embodiments, the degree of modification in a polymer network of a hydrogel of the present invention determines and/or controls at least in part the rate of release of a component (e.g., a protease inhibitor and/or an orexin receptor ligand) that is present in the hydrogel. In some embodiments, a protease inhibitor is present in a first hydrogel and an orexin receptor ligand is present in a second hydrogel, wherein the first hydrogel has a greater degree of modification than the second hydrogel and the first hydrogel has a faster (e.g., greater) rate of release (e.g., faster rate of degradation) than the second hydrogel. In some embodiments, a protease inhibitor and an orexin receptor ligand are each administered (e.g., orally administered) to a subject optionally at the same time, wherein the protease inhibitor and orexin receptor ligand are both present in a hydrogel and the protease inhibitor is released from the hydrogel prior to the orexin receptor ligand, which may reduce or inhibit digestive enzymes prior to release of the orexin receptor ligand and/or protect the orexin receptor ligand (e.g., from degradation by enzymes).

In some embodiments, a method of the present invention comprises administering a composition of the present invention that includes a hydrogel of the present invention, wherein the hydrogel comprises an agent (e.g., an orexin receptor ligand and/or protease inhibitor). In some embodiments, the hydrogel prevents release of the agent in the stomach of a subject and the hydrogel releases the agent in the small intestine of the subject. In some embodiments, the hydrogel falls apart and/or degrades in the intestines. In some embodiments, the hydrogel releases the agent in the stomach of the subject. In some embodiments, the hydrogel falls apart and/or degrades in the stomach. In some embodiments, the hydrogel releases the agents in the prostate of the subject. In some embodiments, the hydrogel falls apart and/or degrades in the prostate. In some embodiments, the hydrogel releases the agent in the peritoneal cavity of the subject. In some embodiments, the hydrogel falls apart and/or degrades in the peritoneal cavity.

In some embodiments, administering a composition of the present invention comprises administering about 0.1 μM to about 10 μM (e.g., about 0.1 μM to about 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, or 10 μM) of an orexin receptor ligand to a subject per week. In some embodiments, administering a composition of the present invention comprises administering about 250,000 kallikrein inactivating units (KIU) to about 15,000,000 KIU (e.g., about 250,000 KIU to about 500,000, 750,000, 1,000,000, 1,500,000, 2,000,000, 2,500,000, 3,000,000, 3,500,000, 4,000,000, 4,500,000, 5,000,000, 5,500,000, 6,000,000, 6,500,000, 7,000,000, 7,500,000, 8,000,000, 8,500,000, 9,000,000, 9,500,000, 10,000,000, 10,500,000, 11,000,000, 11,500,000, 12,000,000, 12,500,000, 13,000,000, 13,500,000, 14,000,000, 14,500,000, 15,000,000) of a protease inhibitor per administration.

In some embodiments, a method of the present invention comprises administering an orexin receptor ligand to a subject via a composition of the present invention, which may treat a cancer in the subject. In some embodiments, administering a composition of the present invention to a subject may treat colorectal cancer, pancreatic cancer, liver cancer, stomach cancer, cervical cancer, brain cancer, adrenal cancer, and/or prostate cancer in the subject. In some embodiments, administering a composition of the present invention to a subject may comprise intravenous injection, intramuscular injection, intracerebroventricular injection, or subcutaneous injection of the composition directly into a tumor and/or tissue surrounding a tumor in the subject. In some embodiments, a composition of the present invention that is configured for injection into a subject (e.g., into a tumor and/or tissue surrounding a tumor) comprises a hydrogel that comprises an orexin receptor ligand and the composition is devoid of a protease inhibitor.

In some embodiments, administering a composition of the present invention to a subject may further comprise administering a chemotherapeutic (e.g., paclitaxel) to the subject. In some embodiments, the chemotherapeutic may be in the same or different composition as a hydrogel comprising an orexin receptor ligand and/or a protease inhibitor of the present invention. In some embodiments, a chemotherapeutic may be administered concurrently with a hydrogel of the present invention. In some embodiments, a chemotherapeutic may be administered sequentially with a hydrogel of the present invention.

In some embodiments, a method of the present invention comprises administering an orexin receptor ligand to a subject via a composition of the present invention, thereby treating a substance abuse disorder in the subject.

In some embodiments, a method of the present invention comprises administering an orexin receptor ligand to a subject via a composition of the present invention, thereby treating a sleep disorder in the subject. In some embodiments, the sleep disorder is insomnia.

In some embodiments, a method of the present invention comprises administering an orexin receptor ligand to a subject via a composition of the present invention, thereby treating a feeding disorder and/or an eating disorder in the subject.

In some embodiments, a method of the present invention comprises administering an orexin receptor ligand to a subject via a composition of the present invention, thereby treating neuropathic pain in the subject.

The present invention is explained in greater detail in the following non-limiting examples.

### EXAMPLES

### Example 1: Determination of the Encapsulated Orexin Release Profile In Vitro

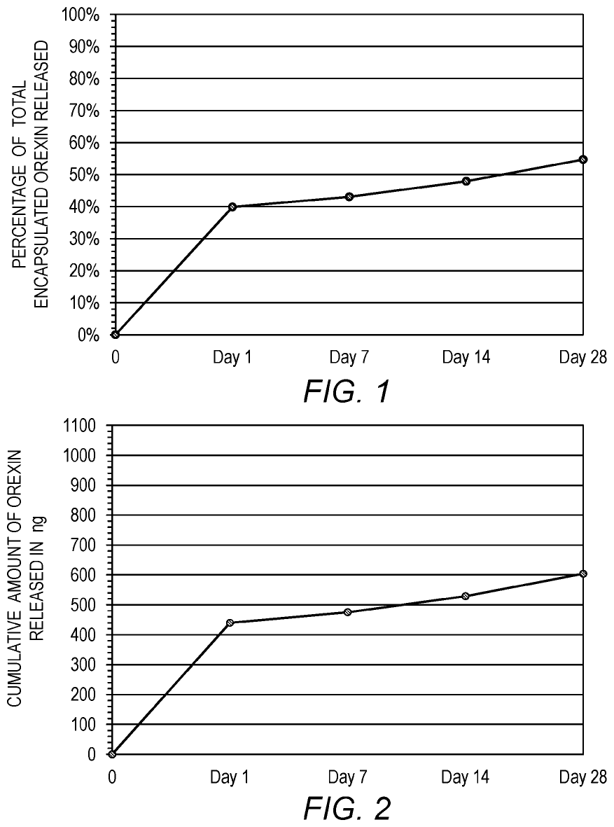

Alginate microbeads were generated using a microfluidic chip device as previously described (Enck K, et al., Design of an Adhesive Film-Based Microfluidic Device for Alginate Hydrogel-Based Cell Encapsulation. Ann Biomed Eng. 2020 March;48(3):1103-1111. doi: 10.1007/s10439-020-02453-9. Epub 2020 Jan. 13. PMID: 31933001). The alginate microbeads were prepared with ultrapurified low-viscosity high-guluronic acid alginate (Sandvika Norway). Orexin was encapsulated within these microbeads by being soaked in a 1000 μg/mL orexin solution while rotating over night at 4° C. After 24 hours, the beads were washed once with 100 mM CaCl₂) and then incubated in 25 mM CaCl₂) dissolved in a 0.9% NaCl solution. Samples were collected over a duration of one month and at set time points: a sample of this solution was taken and the release of orexin was measured using an Enzyme Linked Immunosorbent Assay (ELISA). It was observed that encapsulation of orexin in microbeads (400 microns in diameter) resulted in nearly 60% release of the peptide within a month (FIG. 1), which corresponds to roughly 600 ng of orexin released from the beads (FIG. 2).

### Example 2: Determination of the Effect of Orexin on Pancreatic Tumor (ASPC1 Cells) Inoculated in Nude Mice

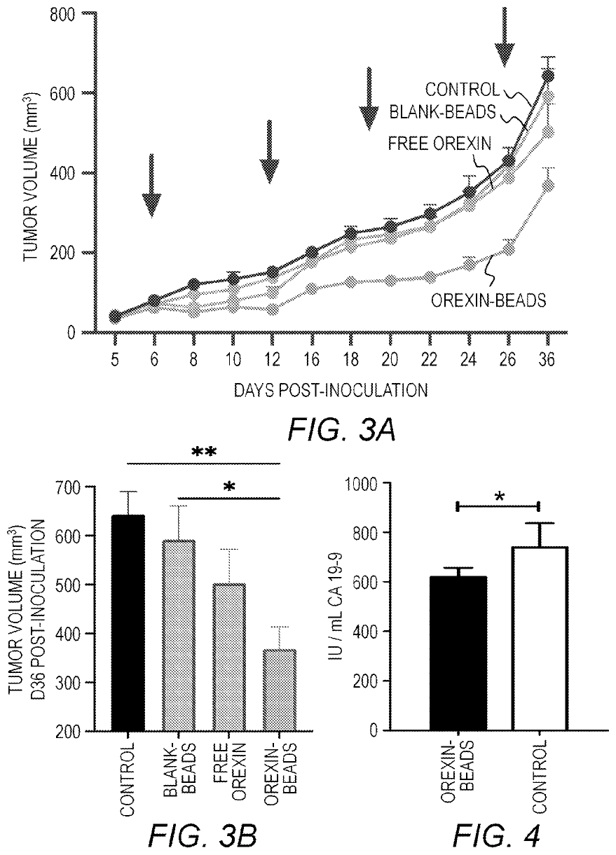

Athymic nude mice were inoculated with ASPC1 pancreatic tumor cells, which are known to highly express the orexin receptor. Six to eight week-old severe combined immunodeficient (SCID) mice housed under pathogen-free conditions were inoculated with 100 μl of 5×10⁶ ASPC1 cells in Matrigel®. The mice were placed into one of four different groups, which were (1) PBS control, (2) orexin alone control, (3) blank bead alone control, and (4) orexin loaded in microbeads, with twelve mice per group (n=12 per group). Intratumoral treatments of either PBS control, free orexin, empty hydrogel microbeads, or orexin encapsulated in hydrogel microbeads were initiated when mice tumors reached a volume of 100 mm³ (~5 days). Four treatments in each of these four groups were performed until day 30 (35 days total including the 5 days post inoculation with the ASPC1 cells), as approved in the animal protocol. Orexin was administered either free or encapsulated in hydrogel microbeads at an intratumoral dose of 0.5 μM in 100 μl saline on day 5 after tumor cell inoculation and once/week until day 30 with daily tumor volume measurements. The other groups were also treated with the relevant medium on the same time schedule as the orexin beads group. The results are shown in FIGS. 3A and 3B. Post day 26, heavy ulceration was observed in tumors of the PBS control, blank bead control, and free orexin control groups. Ulceration led to tumor opening in these groups resulting in tumor mass loss in many mice.

At the end of the study, the animals were bled and plasma samples from the PBS control and the orexin-microbeads were procured and used for measurement of CA19-9 by ELISA (RayBiotech) (FIG. 4). This test measures the amount (e.g., international units per mL (IU/mL)) of a protein called CA 19-9 (cancer antigen 19-9) in a sample of blood. CA 19-9 is a type of tumor marker made by cancer cells or by normal cells in response to cancer in the body.

The foregoing is illustrative of the present invention, and is not to be construed as limiting thereof. The invention is defined by the following claims, with equivalents of the claims to be included therein.

1. A composition comprising:
a first hydrogel; and
an orexin receptor ligand.

2. The composition of claim 1, further comprising a protease inhibitor.

3. The composition of claim 2, wherein the protease inhibitor is in the first hydrogel and/or a second hydrogel.

4. The composition of claim 1, wherein the first hydrogel encapsulates the orexin receptor ligand.

5. The composition of claim 3, wherein the first hydrogel and/or the second hydrogel encapsulates the protease inhibitor.

6. The composition of claim 3, wherein the orexin receptor ligand and protease inhibitor are encapsulated in the same and/or separate hydrogels.

7. The composition of claim 3, wherein the first hydrogel and the second hydrogel are discrete hydrogels.

8. (canceled)

9. (canceled)

10. The composition of claim 1, wherein the first hydrogel has at least one dimension of about 100 microns to about 600 microns.

11. The composition of claim 1, wherein the first hydrogel is a modified alginate hydrogel.

12. The composition of claim 1, wherein the first hydrogel is a crosslinked hydrogel.

13-15. (canceled)

16. The composition of claim 11, wherein the modified alginate of the modified alginate hydrogel is prepared by reacting an alginate with a reactant that is selected from the group consisting of 4-(2-aminoethyl)benzoic acid, 4-(2-aminomethyl)benzoic acid, 4-(2-aminoethyl)aniline, (2-ethylamino) 4-methyl benzene, 4-(2-aminoacetyl)-benzoic acid, 4-(2-aminoethyl)salicylic acid, methyl 4-(2-aminoethyl)benzoate, phenethylamine, 1-[4-(2-aminoethyl)phenyl]ethanone HCl, and/or 4-(2-aminomethyl)aniline and/or esters thereof.

17. The composition of claim 11, wherein the first hydrogel is a 2-amino ethyl or aminomethyl aromatic modified alginate hydrogel.

18. The composition of claim 11, wherein about 0.1% to about 10% of the polysaccharide units of the modified alginate hydrogel are modified.

19. (canceled)

20. The composition of claim 11, wherein the modified alginate of the modified alginate hydrogel comprises at least one unit having a structure of Formula I or Formula II: 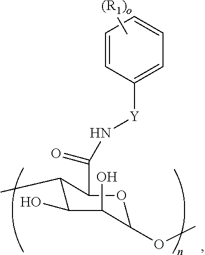
or 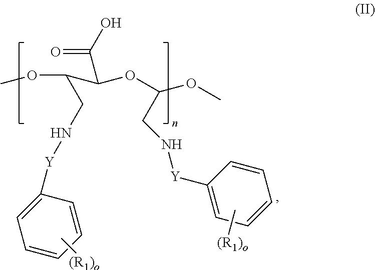
wherein
Y is absent or a C₁-C₄ alkyl or C₁-C₄ alkenyl;
R₁ is each independently selected from the group consisting of —H, —OH, —NH₂, —COOH, —C(O)OR′, —NO₂, —CN, —Br, —Cl, —F, —C₁-C₆ alkylhalide, unsubstituted or substituted —C₁-C₆ alkyl, unsubstituted or substituted —C₁-C₆ alkenyl, —SO₂H, —SO₃H, —COR′ (e.g., —COCH₃), —Si(OH)₃, —SO₂NH₂, —PO(OR′)₂, and —B(OH)₂, wherein R′ is unsubstituted or substituted alkyl, alkenyl, alkynyl, or aryl;
n is from 1 to 1,000,000; and
is 0, 1, 2, 3, 4, or 5.

21. (canceled)

22. The composition of claim 11, wherein the modified alginate of the modified alginate hydrogel comprises oxidized alginate.

23. The composition of claim 1, wherein the orexin receptor ligand is a peptide or protein ligand and/or an orexin antagonist.

24. (canceled)

25. The composition of claim 1, wherein the orexin receptor ligand is present in the first hydrogel in a concentration of about 1 μg of the orexin receptor ligand per mL of the first hydrogel to about 500 μg of the orexin receptor ligand per mL of the first hydrogel.

26. (canceled)

27. The composition of claim 1, further comprising a second therapeutic agent.

28-30. (canceled)

31. The composition of claim 11, wherein the modified alginate of the first hydrogel is present in an amount of about 0.1% to about 10% w/v of the hydrogel.

32-39. (canceled)

40. A method for treating a subject in need thereof, the method comprising:
administering a first hydrogel comprising a therapeutically effective amount of an orexin receptor ligand to the subject, thereby treating the subject.

41-68. (canceled)
